(19) United States
(12) Patent Application Publication
(10) Pub. No.: US 2014/0243830 A1
(43) Pub. Date: Aug. 28, 2014

(54) APPARATUS AND SYSTEMS FOR ROTARY OSTEOTOMY

(71) Applicant: Benjamin A. Baptist, North Riverside, IL (US)

(72) Inventor: Benjamin A. Baptist, North Riverside, IL (US)

(21) Appl. No.: 14/044,724

(22) Filed: Oct. 2, 2013

Related U.S. Application Data
(60) Provisional application No. 61/769,274, filed on Feb. 26, 2013.

(51) Int. Cl. A61B 17/16

(52) U.S. Cl. CPC A61B 17/16
USPC 606/80

(57) ABSTRACT

Disclosed are apparatus and systems for rotary osteotomy that afford increased control of a trajectory angle and direction/re-direction of the tip of an osteotomy apparatus, controllability of the depth of the tip or fine rotation of the device, and tactile feedback. The apparatus includes a tip portion having a first diameter at a first end and a second diameter at a second end larger than the first diameter, wherein at least a part of the tip portion includes a plurality of pitched threads. A shank is fixedly connected to the second end of the tip portion and has at least a third diameter. A handle is fixedly connected to the shank and arranged distal from the tip portion. The handle has a fourth diameter that is at least greater than the second diameter of the tip portion and the third diameter of the shank.

### CLAIM OF PRIORITY UNDER 35 U.S.C. §119

The present Application for Patent claims priority to Provisional Application No. 61/769,274 entitled “Manual Rotary Osteotome” filed Feb. 26, 2013, and hereby expressly incorporated by reference herein.

### BACKGROUND

1. Field

The present disclosure relates generally to apparatus and systems for rotary osteotomy, such as an osteotome or osteotome kit, and more specifically to apparatus and systems for manual rotary osteotomy using a unitary osteotome.

2. Background

An osteotomy is a surgical operation where a bone can be cut, changed in alignment, or bored by cutting and channelization. An osteotomy for placement of a dental implant, for example, consists of making a channel in a bone as a receptor site for a dental fixture to be inserted. In such dental osteotomy, the majority of root-form implants require a round channel which may be prepared with either a drill or an osteotome. The use of osteotomes is preferred by many operators, especially when insufficient bone is present, or the bone is of poor quality in a receptor site. In particular, an osteotome technique allows for the compression and expansion of a receptor site without the removal of bone when forming a channel.

Known osteotomes are typically comprised of a tip portion that effects expansion and compression of bone at a particular bone site. The tip portion may be linear and shaped, such as a conical shape in at least the first part of the tip portion, to advance the osteotome into the bone and effect bone expansion and compression when being driven through impact force applied to the osteotome, such as striking with a mallet. Problems with such devices are that a large impact force is transferred to a patient, and also control of the tip depth, rate of advancement, and trajectory angle or direction is difficult and somewhat unpredictable.

Other known osteotomes includes a threaded tip that effects bone expansion and compression by rotations of the threads thereby pulling the osteotome into the bone as well as compressing the bone to form a channel. An advantage of such osteotomes is greater control over the tip depth as it extends into the bone. Known rotary osteotomes up to this point, however, utilize external driver devices such as a detachable ratchet that extends at a right angle to the rotary axis of the osteotome tip, or a detachable axial drill or motor type driver that engages the osteotome. Such devices may be problematic as there is still difficulty in controlling the angle or direction of the osteotome with such drivers due to either a right angle ratchet, greater length of the driver train, or tolerance play in the engagement mechanism of the driver. Additionally, these osteotomes also may still suffer a degree of depth controllability as the operator cannot easily provide small or slow controlled rotations to advance the osteotome tip, as well as diminished tactile feedback to the operator. Accordingly, a need exists for a rotary osteotome that affords more control of the trajectory angle or direction/re-direction of the tip, as well as affording more controllability of the depth of the tip or fine rotation of the device, as well as tactile feedback.

### SUMMARY

Embodiments disclosed herein address the above stated needs by providing apparatus and systems that provide improved control of the trajectory angle or direction/re-direction of a rotary osteotome device, as well as affording more controllability of the depth of the tip or fine rotation of the device, as well as tactile feedback.

In an aspect, an apparatus for rotary osteotomy is disclosed. The apparatus includes a tip portion having a first diameter at a first end and a second diameter at a second end larger than the first diameter, wherein at least a part of the tip portion includes a plurality of pitched threads. The apparatus further includes a shank fixedly connected to the second end of the tip portion and having at least a third diameter. A handle is fixedly connected to the shank and disposed distal from the tip portion, the handle having a fourth diameter that is at least greater than the second diameter of the tip portion and the third diameter of the shank.

In another aspect, an osteotomy system is disclosed. The system includes a plurality of osteotomy apparatus. Each apparatus comprising includes a tip portion having a first diameter at a first end and a second diameter at a second end larger than the first diameter, wherein at least a part of the tip portion includes a plurality of pitched threads, a shank fixedly connected to the second end of the tip portion and having at least a third diameter, and a handle fixedly connected to the shank and disposed distal from the tip portion, the handle having a fourth diameter that is at least greater than the second diameter of the tip portion and the third diameter of the shank. In another aspect, at least the first and second diameters of a tip portion of a first osteotomy apparatus of the plurality are smaller than the corresponding first and second diameters of a tip portion of at least a second osteotomy apparatus of the plurality.

### BRIEF DESCRIPTION OF THE DRAWINGS

FIG. 1 illustrates a side view of an exemplary osteotomy apparatus according to the present disclosure.

FIG. 2 illustrates a side view of the tip portion of the osteotomy apparatus exemplified in FIG. 1.

FIGS. 3a, 3b, 3c, 3d, 3e, 3f, 3g, 3h, and 3i illustrates various sequential steps in an exemplary operation of the osteotomy apparatus illustrated in FIGS. 1 and 2 for a dental procedure; namely expansion of a narrow alveolar ridge for subsequent placement of an implant in the expanded ridge.

FIGS. 4a, 4b, 4c, 4d, 4e, 4f, and 4g illustrate another exemplary operation of the osteotomy apparatus illustrated in FIGS. 1 and 2 for a type of dental procedure; namely a tooth extraction and subsequent placement of a dental implant

FIGS. 5a, 5b, 5c, 5d, 5e, and 5f illustrate yet one more exemplary operation of the osteotomy apparatus illustrated in FIGS. 1 and 2 for another type of dental procedure; namely a sinus elevation and subsequent implant placement.

### DETAILED DESCRIPTION

The presently disclosed apparatus and systems provide a solution to above-described need. In particular, an osteotome apparatus and system is disclosed that does not require the use of a mallet to advance the instrument into a bone, allows an operator to control the rate of advance and the rate of expansion of the osteotomy, and provides greater control and accuracy of the trajectory angle and direction or redirection of the osteotome. Furthermore, the disclosed apparatus and system afford the ability to elevate the floor of the maxillary sinus with a greater degree of control when compared to conventional mallet style osteotomes. Additionally, the presently disclosed system includes a system or protocol for developing an osteotomy that has been developed, which allows an operator to efficiently and systematically develop an osteotomy, suitable for receiving root form implants from a variety of manufacturers. In particular, the system or protocol uses a plurality or series of osteotomes that graduate in dimension size. Both the unique features of the instrument and the protocol, allow for additional osteotomes of varied dimensions to be developed.

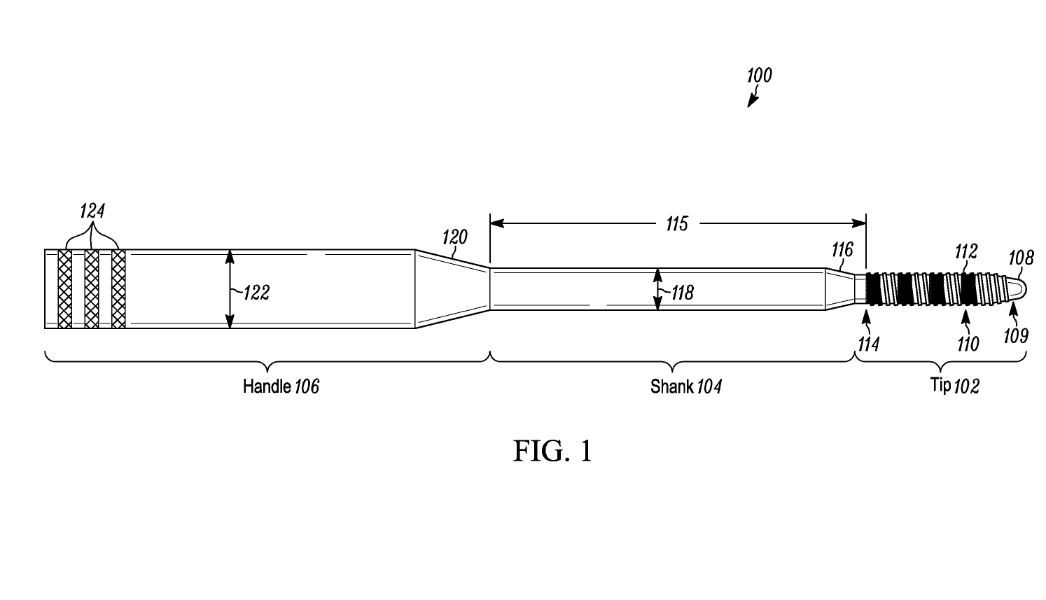

FIG. 1 illustrates a side view of an exemplary osteotomy apparatus 100 according to the present disclosure. Apparatus 100 consists of a generally cylindrical and linear device having at least 3 distinct portions; a tip portion 102, a shank 104, and a handle 106. Apparatus 100 may be constructed as a unitary body, but is not limited to such, and could instead, for example, consist of separate portions that may be assembled together to form a unitary body when assembled. The tip portion 102 further includes a smaller diameter portion 108 at a front or first end 109 of the tip 102 that increases in diameter toward a start 110 of a main portion 112 having a greater diameter portion than the smaller diameter portion 108 of the tip 102. The tip 102 further includes threads to convert rotational motion of the apparatus 102 into linear motion. The threads may extend from the front end 109 to a distal or second end 114 of the tip portion 102, or any portion thereof, such as the threads starting at a distance from front end 109 as illustrated in the example of FIG. 1.

It is noted that the first end 108 may be shaped to point (not shown) or rounded as shown in FIG. 1. According to an aspect, a pointed end for first end 109 may be utilized for certain smaller diameters of the tip portion 102, whereas a rounded end for the first end 109 may be utilized with other diameters that are larger than the certain smaller diameters. These different topologies for the first end shape may be helpful when multiple sizes of the apparatus 100 are used in a sequential fashion from smaller to larger when gradually enlarging a osteotomy site in a bone. That is, a sharper tipped apparatus is used for starting an osteotomy site, while the more rounded apparatus are used for subsequent enlargement of the osteotomy site through further compression of the bone.

A middle portion of the apparatus 100 comprises a shank 104 of a particular length or distance 115 and disposed between the tip portion 102 and the handle 106. Shank 104 may include a tapered portion 116 that increases the shank 104 diameter from the diameter of the end part 114 of the tip portion 102 to a particular diameter 118 that is greater than the diameter of tip 102 portion, as illustrated. It is noted that the tapering to increase diameter of the shank 104 is only exemplary, and that the diameter of shank 104 could also be substantially the same of the greatest diameter of the tip portion 102, or even less as well. In an aspect, however, the shank diameter would typically include the tapered portion 116 to provide an intermediate increase of diameter along the linear axis of the apparatus 100 prior to the handle 106, which is typically even larger in diameter. Additionally, length 115 of shank 104 affords length between the tip portion 102 and the handle 106 to afford leverage to an operator of apparatus 100, such as for controlling direction/trajectory angle or lateral force, if necessary. The particular measurement of length 115 is varied and may be on various factors including, but not limited to, the presence (or lack) of tapered portions (e.g., 116), the diameter of the tip portion 102, and the type of osteotomy procedure being performed.

Handle 106 is disposed linearly distal from the tip portion 102. Handle 106 may include a tapered portion 120 to increase the diameter of the apparatus 100 from the diameter 118 of the shank 104 to a greater diameter 122, although the apparatus is not limited to such construction. The handle 106 is configured to be manually grasped by an operator to allow for manual application of rotational force to afford rotation of the tip portion 102 and threads 112 for rotary osteotomy, as well as for the application of force in the direction of the linear axis and to guide the angle and direction of the apparatus 100 when in use. To allow an operator's hands or fingers to get a better grip on the apparatus 100, the surface of handle 106 may include one or more textured portions for gripping, such as knurled portions 124, which may be annular as shown in FIG. 1 or, alternatively, continuous throughout at least a portion of the handle 106. In an alternative, the cross sectional topology of the handle may be configured to have a flattened portion allowing for increased grip of the handle 106.

Due to the nature of apparatus 100 for clinical use in osteotomy or other similar medical or dental procedures in bone, apparatus 100 may typically constructed of a rigid and durable material, such as stainless steel or titanium. Notwithstanding, in light of continuing improvements in metallurgy and composites, apparatus 100 may be made from any alternative material that meets the requirements of clinical utility and provides rigidity and durability.

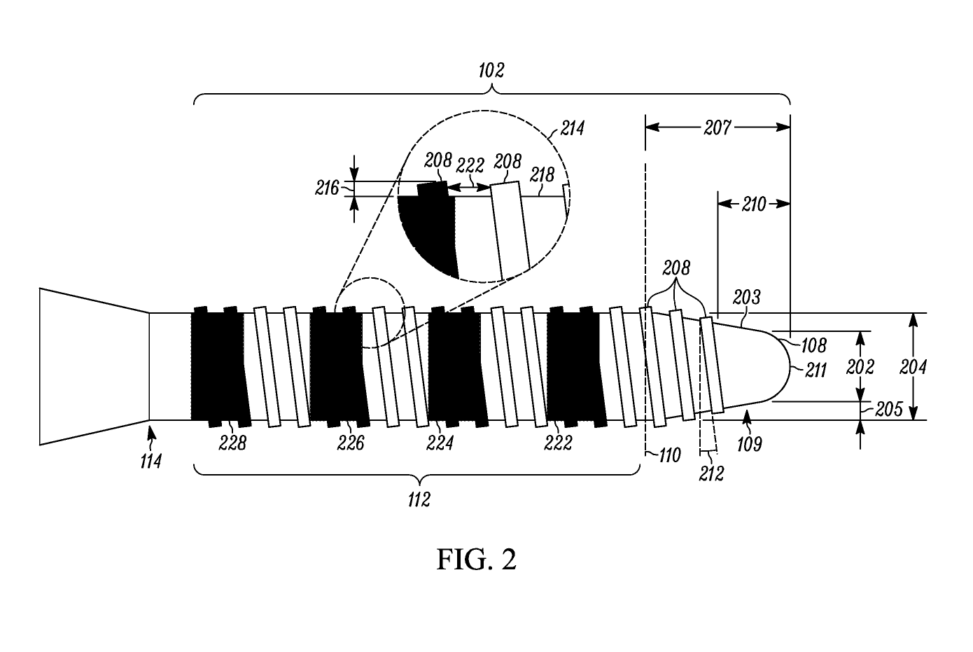

FIG. 2 illustrates a side view of the tip portion 102 of the osteotomy apparatus 100 exemplified in FIG. 1. The first end portion 109 includes a smaller diameter portion 108 that has a particular diameter 202. In various specific examples, diameter 202 may be from approximately 0.1 millimeters up to approximately 3.7 millimeters dependent on the desired size of a particular osteotomy site, and to accommodate for various sizes of the tip portion 102. The diameter may taper in a tapered portion 203 to a diameter 204 starting at the start 100 of the main portion 112. In various examples, diameter 204 may be from approximately 2.3 millimeters up to approximately 4.4 millimeters dependent on the desired size of a particular osteotomy site, and to accommodate for various sizes of the tip portion 102.

The rate of tapering of the tapering portion 203 may be linear and uniform as illustrated, increasing the diameter by a distance 205 over a length 207. A constant, linear increase of the diameter of tapering portion 203, however, is not limiting and other topologies of a non-linear rate of tapering may also be contemplated.

As discussed before, the tip 102 includes a plurality of pitched threads 208 that advance the apparatus 100 linearly along the axis of rotation when the apparatus is rotated. In an aspect, the threads start a distance 210 from the apex 211 of the first end 108, thus allowing the first end portion 109 to be free of threading. In another aspect, however, it contemplated that the threading could start from the apex 211 for certain applications. The plurality of threads 208 are pitched at a prescribed thread angle 212.

As illustrated by expanded view 214, each of the plurality of threads 208 may be configured to have a prescribed thread depth 216 above an outer diameter surface 218 of the tip 102. The surface 218 may be part of the main portion 112, as illustrated, but could also be a representation of the surface of the tapered portion 203, as well. As may also be seen in expanded view 214, the pitch 220 is set to achieve a predetermined linear distance of advancement of the apparatus 100 for each full rotation or revolution (i.e., 360 degrees) of the apparatus 100 (i.e., the “lead”). For example, the pitch 220 could be set such that the lead or linear advancement of the apparatus 100 is 1 mm for each complete revolution of the apparatus 100.

In order to allow an operator a measure of the distance of advancement of the apparatus 100 into an osteotomy site, tip portion 102 may also include a plurality of markings 222, 224, 226, 228. In one example, the markings 222, 224, 226, and 228 may each have width of 2 millimeters and be spaced 2 millimeters apart. It will be appreciated by those skilled in the art that the markings could be greater in number to provide finer degrees of measurement, or, alternatively, less if fine degree of measurement is not needed. The markings may be placed by any means, such laser marking, for example.

In operation, apparatus 100 may be used to provide access to the bone where an osteotomy is desired by means of reflecting the tissue in part or total, but is not necessarily limited to such. It is noted that, in at least one aspect, tissue deflection of apparatus 100 in FIGS. 1 and 2 is effected by the larger diameter 204 of the tip 102. Thus, in an aspect threads 208 substantially serve only to linearly advance the apparatus, and do not effectuate any meaningful or efficacious reflecting of bone tissue. It is also noted that when performing an osteotomy, interstitial fluid present around bone cells is compressed and time where the osteotome is temporarily stopped from advancing is needed to allow this pressure to dissipate before continuing the osteotomy. The present manual osteotomy apparatus allows tactile feedback to the operator to more accurately sense when the interstitial fluid pressure is dissipated and also to more accurately judge when to stop turning the apparatus 100 and when to continue.

According to another aspect of a system using a plurality of various diameter and shaped osteotomy apparatus as described above, a plurality of apparatus may be used to gradually increase the bore size of osteotomy site in a bone. In particular, a first apparatus would have the smallest diameters (e.g., diameters 202 and 204). Furthermore, the first apparatus may have an apex 211 shape that is substantially pointed, whereas the larger second apparatus and so on would have substantially rounded apexes.

In another aspect, the smaller osteotomes may be used either directly to pierce the cortical plate and medullary bone or in combination with a burr of similar size, and then by exerting a vector of force along the axis of the instrument while rotating the handle to advance the threads. Upon penetration the operator may redirect the trajectory of the instrument via manipulation of the handle (e.g., 106 in FIG. 1) to which this device is rigidly connected as the device advances through continued rotation. Once a depth and trajectory is established by the operator, the instrument is removed from the initial osteotomy, and the next larger graduation of the instrument design is inserted and sequentially advanced to the depth determined by the operator. Minor corrections in trajectory may be accomplished with the graduating osteotomes at the discretion of the operator. This sequence will establish an osteotomy of adequate depth, diameter, and trajectory suitable for the insertion of a dental implant, with the finalization and closure of the surgical site at the discretion of the operator. It is noted that the dental implant may include, but is not limited to, Zimmer™, Nobel Biocare™, Biohorizons™, and Implant Direct Sybron™, as merely a few examples.

An alternative manner in which this device may be used, but not limited to, is to provide access to the bone by introducing the finest of the instruments claimed here through the cribriform plate of a fresh extraction socket and into the medullary bone with or without the aid of a bun; and then by exerting a vector of force along the axis of the instrument while rotating the handle to advance the threads. Upon penetration, the operator may redirect the trajectory of the instrument via manipulation of the handle 106, as the apparatus 100 linearly advances through continued rotation. Once a depth and trajectory is established by the operator, the instrument is removed from the initial osteotomy, and a next larger graduation of the apparatus is inserted and sequentially advanced to the depth determined by the operator. Minor corrections in trajectory may be accomplished with the graduating osteotomes at the discretion of the operator. This sequence will establish an osteotomy of adequate depth, diameter, and trajectory suitable for the insertion of a dental implant, with the finalization and closure of the surgical site at the discretion of the operator.

In a further aspect, it is noted that the smallest, first diameter of the tip portion of each successive instrument is smaller than the larger, second diameter of the tip portion of its predecessor. For example, the smaller diameter of the tip portion (i.e., the diameter corresponding to diameter 202 in FIG. 2) for a second, successive instrument will be smaller or less than the larger diameter portion (i.e., the diameter corresponding to diameter 204 in FIG. 2) that for the previous instrument. As those skilled in the art will appreciate, this sizing of tip portions for successively applied instruments will afford optimal performance for the incremental successive enlargement of an osteotomy site.

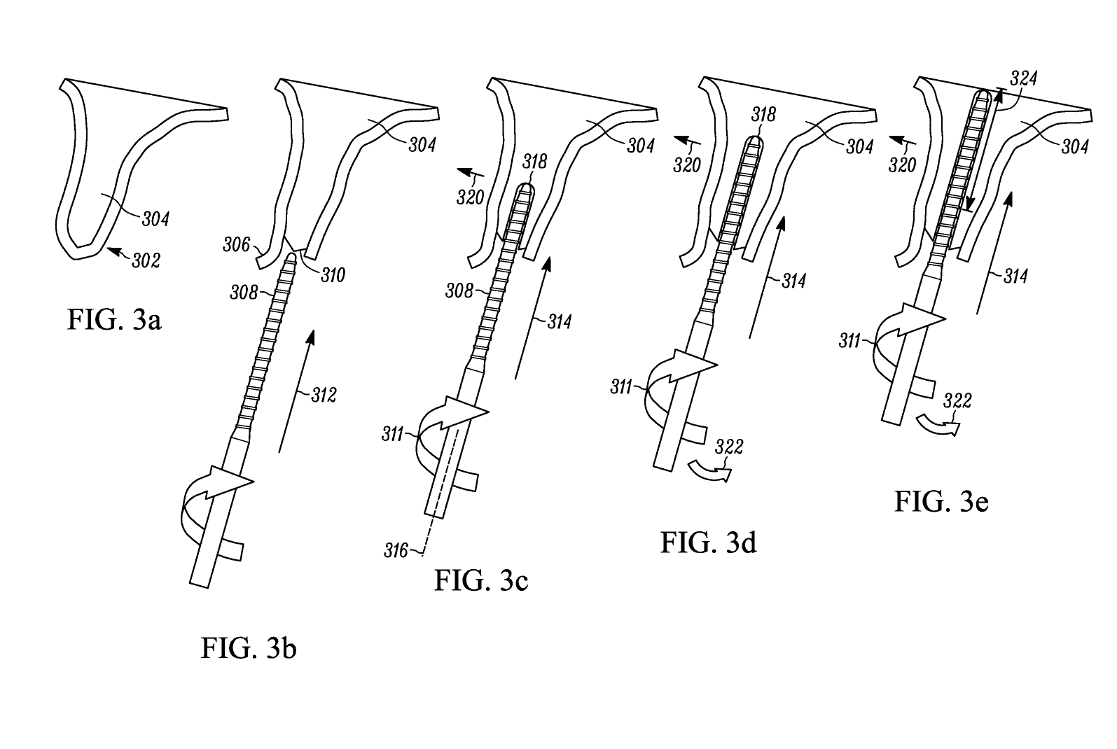

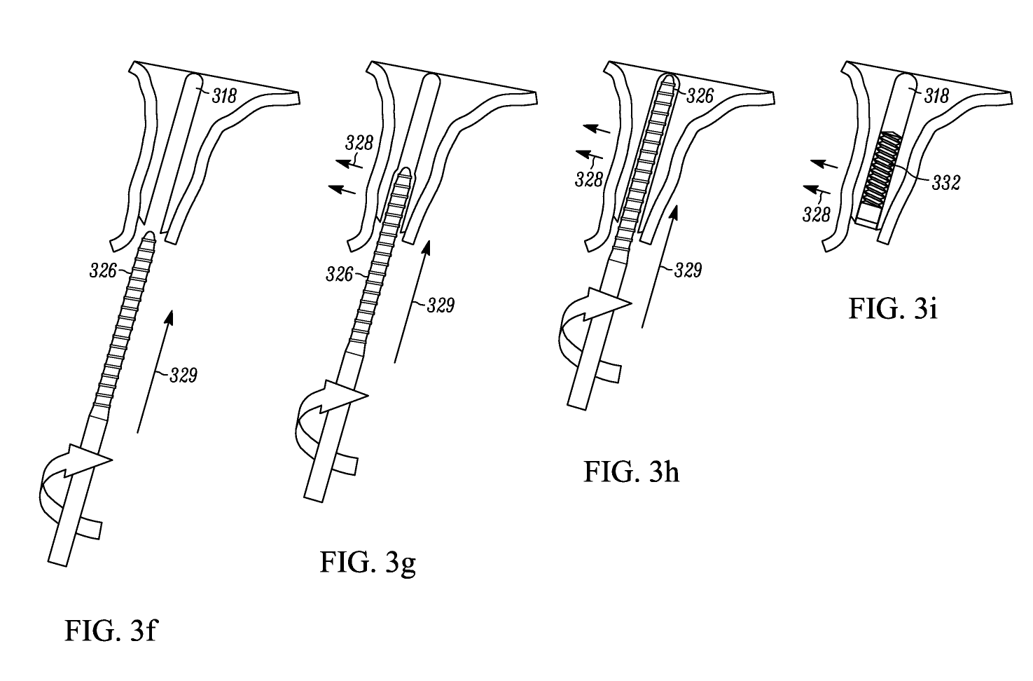

FIGS. 3a through 3i illustrates various sequential steps in an exemplary operation of the osteotomy apparatus illustrated in FIGS. 1 and 2 for a dental procedure; namely expansion of a narrow alveolar ridge for subsequent placement of an implant in the expanded ridge. FIG. 3a illustrates a site of 302 that has been identified as an implant receptor site, but has insufficient Labio-Lingual dimension; i.e., a narrow ridge. Access to the underlying bone 304 may be first obtained via reflection/removal of the overlying gingiva and periosteum (i.e., the membrane that lines the outer surface of a bone) collectively shown as element 306 as illustrated in FIG. 3b. Using the apparatus of FIGS. 1 and 2 (denoted as apparatus 308, which is not necessarily shown to scale and is not necessarily intended to illustrate the entire apparatus, but merely is intended to illustrate the tip and shank portions and how they engage with and relative to a surgery site for FIGS. 3b-3i), for example, the cortical plate 310 may be punctured with the apparatus with application of force 312. FIG. 3c illustrates that the apparatus 308 may be advanced by rotational force 311 on the entire apparatus 308 as an apical vector of force 314 (i.e., a force vector having a direction towards the root tip of a tooth) is applied along the long axis 316 of the apparatus 308 such that the tip or apex of apparatus 308 is advanced into the bone 304. FIG. 3c further illustrates that osteotomy or bore 318 results from the compression force 320 resultant from the apparatus 308 being advanced into the physiologic body (e.g., bone 304). It should be noted that the osteotomy is not the result of any cutting by apparatus 308, but rather due to compression force (i.e., force 320) caused by displacement as the apparatus is advanced into the bone 304 via rotation of the apparatus.

FIG. 3d further illustrates further advancement of the apparatus 308 into bone 304, as well as the feature of redirection of planned osteotomy may be achieved by exerting additional force 322 in the palatal direction of the handle of apparatus 308. Through roughly Class 1 lever mechanics (i.e., a fulcrum located somewhere between the two ends of apparatus 308), the application of lateral or labial force 322 effectuates redirection of the tip of the instrument labially as it is also advanced with continued apical 314 and rotational force 311. As illustrated in FIG. 3e, the apparatus is advanced to a final desired vertical depth 324 through the application of the apical force 314 and rotational force 311.

In an aspect, if the diameter of bore 318 is too small for a particular implant, at least one next larger sized apparatus 326 in a series of one or more further osteotomy apparatus of varying and larger diameters may be introduced in order to sequentially enlarge the diameter of the osteotomy 318 to a desired final diameter. The series of FIGS. 3f, 3g, and 3h illustrate one example of this further enlargement and further application of compression forces 328 on the bone 304 through apical force 329 applied on the apparatus 326 and rotational force 311.

As a final step, a dental implant 332 is inserted in the osteotomy 318 as illustrated in FIG. 3i. The implant 332 insertion may be based on the particular manufacture/clinical recommendations for finalization of surgical site set by whatever manufacture of the implant.

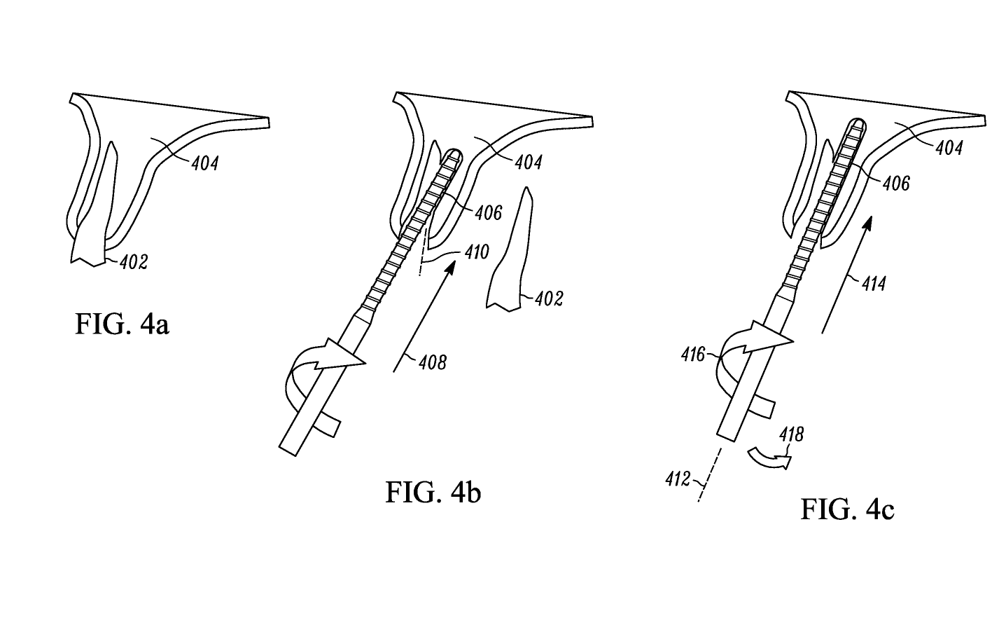

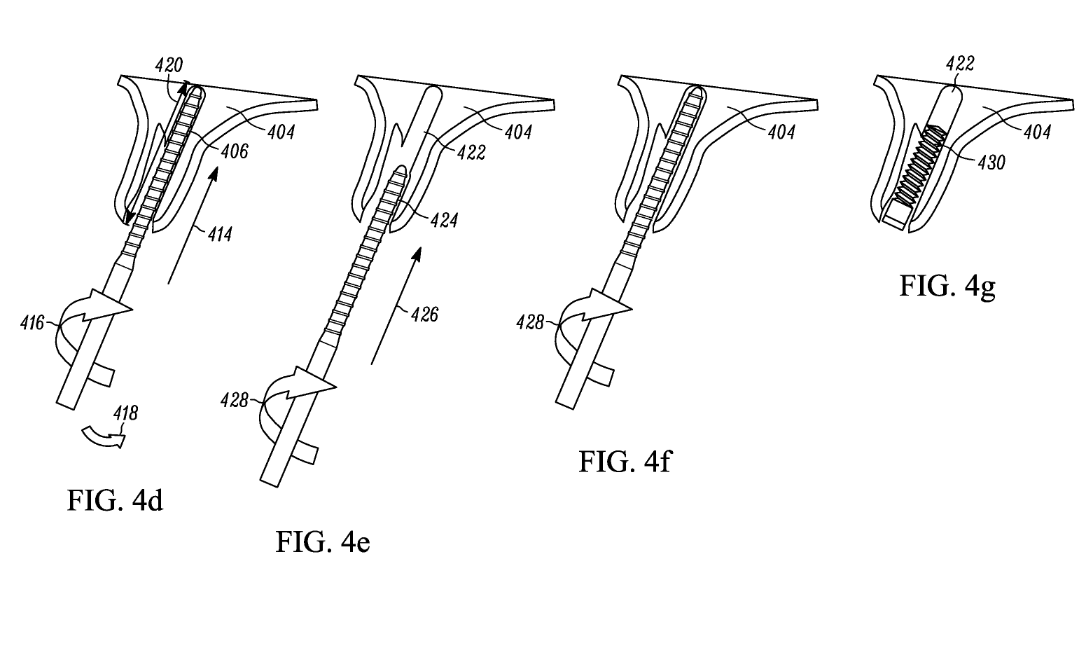

FIGS. 4a through 4g illustrate another exemplary operation of the osteotomy apparatus illustrated in FIGS. 1 and 2 for a type of dental procedure; namely a tooth extraction and subsequent placement of a dental implant. As illustrated in FIG. 4a, a non-salvageable tooth 402 is identified with adequate bone 404 to be able to stabilize a dental implant. As illustrated in FIG. 4b, after the tooth 402 is extracted in order to preserve surrounding tissue (i.e., extracted atraumatically), the osteotomy apparatus 406, which is exemplified by the apparatus illustrated in FIGS. 1 and 2, may be used to puncture the cribriform plate. It is noted here that apparatus 406 is not necessarily shown to scale and is not necessarily intended to illustrate the entire apparatus, but merely is intended to illustrate the tip and shank portions of each apparatus and how they engage with and relative to a surgery site for FIGS. 4b-4g. The apparatus 406 is typically several millimeters coronal to the original root apices and engaging the palatal wall to avoid pressure on the buccal plate as indicated by the angled approach of apparatus 406 and vector force 408 relative to the original root apices indicated by line 410 as illustrated in FIG. 4b.

Once the cribriform plate is pierced, FIG. 4c illustrates that the apparatus is advanced and redirected along the desired final orientation as illustrated by axis 412. The advancement is achieved with apical vector force 414 and rotational force 416. In an aspect, further lateral force 418 may be applied to the handle portion of apparatus 406 to effectuate a fulcrum for direction and redirection of the tip of apparatus 406.

In FIG. 4d, a redirected apparatus 406 is advanced to a desired final depth 420 through continued application of vector force 414, rotational force 416, and lateral force 418, if needed for further adjustments for redirection. In an aspect, if the diameter of osteotomy 422 is too small for a particular implant, at least one next larger sized apparatus 424 in a series of one or more further osteotomy apparatus of varying and larger diameters may be introduced in order to sequentially enlarge the diameter of the osteotomy 422 to a desired final diameter. The series of FIGS. 4e and 4f illustrate one example of this further enlargement and further application of compression of the bone 404 through apical force 426 and rotational force 428 applied to the apparatus 424. As a final step illustrated in FIG. 4g, a dental implant 430 is inserted into the osteotomy site 422, based on prescribed manufacturer/clinical recommendations for finalization of surgical site.

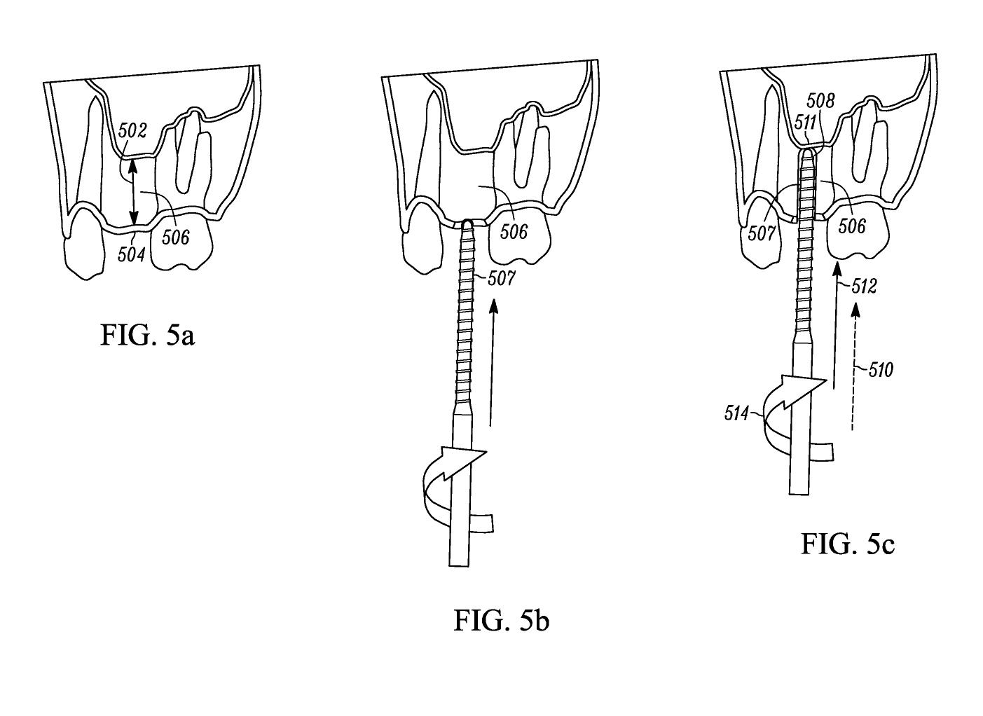

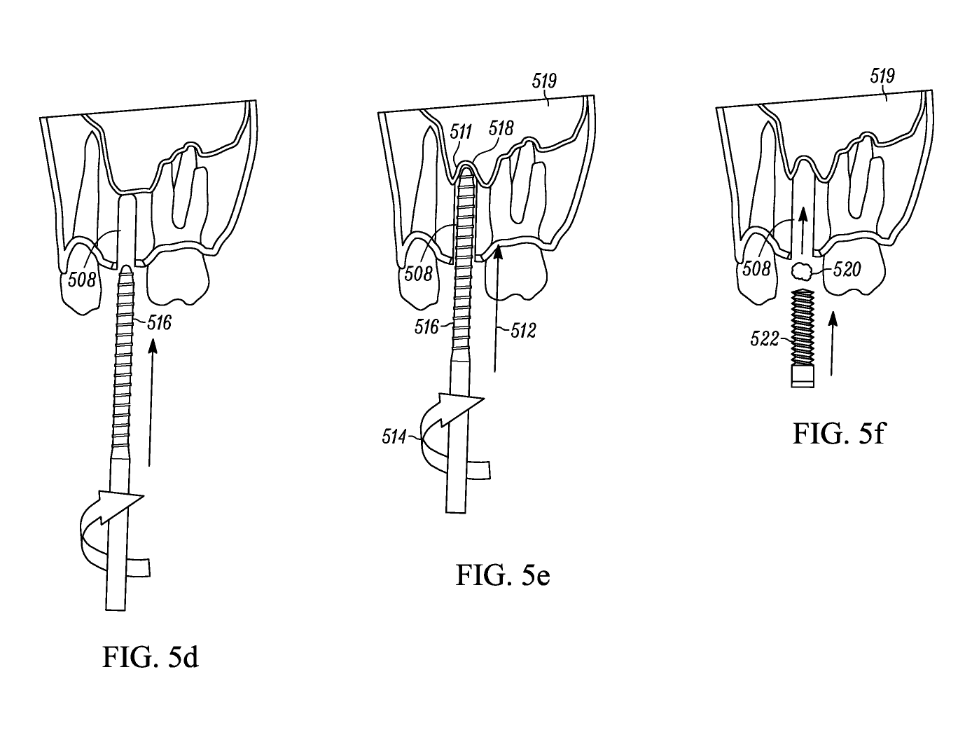

FIGS. 5a-5f illustrate yet one more exemplary operation of the osteotomy apparatus illustrated in FIGS. 1 and 2 for another type of dental procedure; namely a sinus elevation and subsequent implant placement. FIG. 5a is illustrative of a subantral intraoral site having an inadequate vertical boney dimension 502. As illustrated by FIGS. 5a and 5b, access to bone 506 for an osteotomy apparatus 507 configured in accordance with the example of FIGS. 1 and 2 is obtained via reflection or removal of the overlying gingiva and periosteum 504. It is noted that apparatus 507 is not necessarily shown to scale and is not necessarily intended to illustrate the entire apparatus, but merely is intended to illustrate the tip and shank portions of each apparatus and how they engage with and relative to a surgery site for FIGS. 5b-5e.

FIG. 5c illustrates that an initial sized osteotomy 508 is prepared along a desired final orientation (indicated by line 510) to the depth of the sinus/antral floor 511. The preparation is accomplished by advancement of apparatus 507 into bone 506 through application of axial force 512 and rotational force 514.

FIGS. 5d and 5e illustrate that the osteotomy 508 may be sequentially enlarged with one or more additional apparatus 516 larger than the previous apparatus to a desired final diameter. It is further noted that during advancement with the final or largest apparatus 516 as illustrated specifically in FIG. 5e, the apparatus 516 may then be further advanced by linear and rotational forces 512, 514 to achieve an up-fracture 518 of the sinus floor 511, and safely elevate the sinus membrane 519 to a desired vertical height (e.g. 2-3 mm) without tearing it. In a final step illustrated in FIG. 5f, a mass or bolus 520 of a grafting material, such as collagen may be inserted into the osteotomy site 508 before inserting a dental implant 522.

It is noted that the word “exemplary” as used herein means “serving as an example, instance, or illustration.” Thus, any embodiment or example described herein as “exemplary” is not necessarily to be construed as preferred or advantageous over other embodiments.

It is understood that the specific order or hierarchy of steps in the processes disclosed is merely an example of exemplary approaches. Based upon design preferences, it is understood that the specific order or hierarchy of steps in the processes may be rearranged while remaining within the scope of the present disclosure. The accompanying method claims present elements of the various steps in a sample order, and are not meant to be limited to the specific order or hierarchy presented.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. An apparatus for rotary osteotomy comprising:
a tip portion having a first diameter at a first end and a second diameter at a second end larger than the first diameter, wherein at least a part of the tip portion includes a plurality of pitched threads;
a shank fixedly connected to the second end of the tip portion and having at least a third diameter; and
a handle fixedly connected to the shank and disposed distal from the tip portion, the handle having a fourth diameter that is at least greater than the second diameter of the tip portion and the third diameter of the shank.

2. The apparatus as defined in claim 1, wherein the tip portion includes a tapered portion at the first end that tapers the diameter of the tip portion from the first diameter to the second diameter.

3. The apparatus as defined in claim 1, wherein the plurality of pitched threads are configured such that at least a portion of the plurality of pitched threads have the prescribed thread depth extending beyond at least one of the first diameter and the second diameter.

4. The apparatus as defined in claim 1, wherein the plurality of pitched threads start a prescribed distance from the first end of the tip portion.

5. The apparatus as defined in claim 1, wherein the plurality of pitched threads are configured with a prescribed pitch to advance the apparatus a predetermined linear distance per rotation of the apparatus.

6. The apparatus as defined in claim 1, wherein the shank is configured with a predetermined length creating a mechanical moment to effectuate a prescribed degree of leverage for the tip portion.

7. The apparatus as defined in claim 1, further comprising a plurality of markings on the tip portion at predetermined distances apart to allow measurement of linear travel of the apparatus.

8. The apparatus as defined in claim 1, wherein the handle includes one of one or more textured portions and a flattened portion to allow increased grip of the handle

9. The apparatus as defined in claim 1, wherein an apex of the tip portion located at the first end is configured substantially as a point.

10. The apparatus as defined in claim 1, wherein an apex of the tip portion located at the first end is substantially rounded.

11. An osteotomy system comprising:
a plurality of osteotomy apparatus, each apparatus comprising:
a tip portion having a first diameter at a first end and a second diameter at a second end larger than the first diameter, wherein at least a part of the tip portion includes a plurality of pitched threads;
a shank fixedly connected to the second end of the tip portion and having at least a third diameter; and
a handle fixedly connected to the shank and disposed distal from the tip portion, the handle having a fourth diameter that is at least greater than the second diameter of the tip portion and the third diameter of the shank;
wherein at least the first and second diameters of a tip portion of a first osteotomy apparatus of the plurality are smaller than the corresponding first and second diameters of a tip portion of at least a second osteotomy apparatus of the plurality.

12. The system as defined in claim 11, wherein an apex of the tip portion located at the first end of the first osteotomy apparatus is configured substantially as a point, and an apex of the tip portion located at the first end of the second osteotomy apparatus is substantially rounded.

13. The system as defined in claim 11, wherein the tip portion for each of the plurality of osteotomy apparatus includes a tapered portion at the first end that tapers the diameter of the tip portion from the first diameter to the second diameter.

14. The system as defined in claim 13, wherein the first diameter of the tip portion of each successive instrument in the plurality of osteotomy apparatus is configured to be sized smaller than the second diameter of the tip portion of a preceding instrument in the plurality of osteotomy apparatus.

15. The system as defined in claim 11, wherein the plurality of pitched threads for each of the plurality of osteotomy apparatus are configured such that at least a portion of the plurality of pitched threads have the prescribed thread depth extending beyond at least one of the first diameter and the second diameter.

16. The system as defined in claim 11, wherein the plurality of pitched threads for each of the plurality of osteotomy apparatus start a prescribed distance from the first end of the tip portion.

17. The system as defined in claim 11, wherein the plurality of pitched threads for each of the plurality of osteotomy apparatus are configured with a prescribed pitch to advance the apparatus a predetermined linear distance per rotation of the apparatus.

18. The system as defined in claim 11, wherein the shank for each of the plurality of osteotomy apparatus is configured with a predetermined length creating a mechanical moment to effectuate a prescribed degree of leverage for the tip portion.

19. The system as defined in claim 11, each of the plurality of osteotomy apparatus further comprising a plurality of markings on the tip portion at predetermined distances apart to allow measurement of linear travel of the apparatus.

20. The system as defined in claim 11, wherein the handle for each of the plurality of osteotomy apparatus includes one of one or more textured portions and a flattened portion to allow increased grip of the handle.
